(19) United States
(12) Patent Application Publication
(10) Pub. No.: US 2025/0195140 A1
(43) Pub. Date: Jun. 19, 2025

(54) METHOD AND SYSTEM FOR DISTINGUISHING BETWEEN STONE AND TISSUE WITH A LASER

(71) Applicant: Lumenis Ltd., Yokneam Illit (IL)

(72) Inventors: Vitaly Rondel, Hadera (IL); Hernan Altman, Tivon (IL)

(73) Assignee: Lumenis Ltd., Yokneam Illit (IL)

(21) Appl. No.: 18/956,087

(22) Filed: Nov. 22, 2024

Related U.S. Application Data
(60) Provisional application No. 63/602,122, filed on Nov. 22, 2023.

(51) Int. Cl. A61B 18/20; A61B 18/00

(52) U.S. Cl. CPC A61B 18/20; A61B 2018/00505; A61B 2018/00904; A61B 2018/00982; A61B 2018/205547

(57) ABSTRACT

The present disclosure provides a method and system for distinguishing between a stone and a tissue based on fluorescence from the stone or the tissue. Multiple reference beams are projected on a target to determine the distance to the target. The intensity of a fluorescence excitation beam is measured in conjunction with the determined distance to calculate the luminescence of the target and therefore it is a valid target for therapeutic lasing. In case a valid target for therapeutic lasing is detected and a switch is actuated, the therapeutic lasing will be enabled. In case a valid target for therapeutic lasing is not detected and the switch is actuated, the therapeutic lasing will be immediately disabled. The fluorescence excitation beam is pulsed at a high intensity and low duty cycle to avoid blinding an endoscopic imager used in the procedure.

### CROSS REFERENCE TO RELATED APPLICATIONS

This application claims the benefit of U.S. Provisional Patent Application Ser. No. 63/602,122 filed on Nov. 22, 2023, the disclosure of which is incorporated herein by reference.

### TECHNICAL FIELD

The present disclosure generally relates to optical systems and optical fibers used in medical or therapeutic laser treatments. Particularly, but not exclusively, the present disclosure relates to a method and system for distinguishing between a urinary stone and surrounding tissue.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, the stones are required to be fragmented into smaller pieces. A technology known as laser lithotripsy may be used for such fragmenting processes, wherein for small to medium sized urinary stones, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it. In another instance, a laser and optic fiber technology is used in coagulation or ablation treatments. During an ablation treatment, laser light is delivered to the tissue to vaporize the tissue. During a coagulation treatment, laser light is used to induce thermal damage within the tissue. Such ablation treatments may be used for treating various clinical conditions, such as Benign Prostate Hyperplasia (BPH), cancers such as prostate cancer, liver cancer, lung cancer and the like, and for treating cardiac conditions by ablating and/or coagulating a part of the tissue in the heart.

These treatments which use laser and optic fiber technology require high amounts of accuracy to ensure that the laser is aimed at the right target (stone, tissue, tumor etc.), to achieve the clinical objective of tissue ablation, coagulation, stone fragmentation, dusting, and the like. However, during a laser lithotripsy procedure, the visibility is often reduced due to a turbid water environment. Due to the lack of visibility in the treatment environment, a physician may mistakenly aim (or activate) the laser at tissue instead of stone resulting in unwanted damage the patient's tissue. Thus, there is a need to distinguish between tissue and stone.

Techniques to distinguish tissue and stone include the use of an excitation beam at wavelengths under which the stone fluoresces more than the surrounding tissue.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

This disclosure provides design, material, manufacturing method, and use alternatives for medical devices and medical systems. In a first example, a system includes a first laser source to generate a first reference laser beam of a first wavelength and a second reference laser beam at a second wavelength; a second laser source to generate a fluorescence excitation laser beam of a third wavelength; an optical fiber having a distal end and a proximal end, the optical fiber configured to receive laser light from the first and second laser sources at the proximal end, to emit the laser light out of the distal end, and to receive reflected laser light into the distal end; a reference light detector to measure intensity of the reflected light at the first and second wavelengths; a fluorescence light detector to measure intensity of light at a fourth wavelength fluoresced from a target in response to the emission of the fluorescence excitation laser beam; and a processor and memory comprising instructions that when executed by the processor cause the processor to determine a distance from the distal end of the optical fiber to the target based on the measured intensity of the reflected light at the first and second wavelengths and determine whether a fluorescence of a target is greater than or equal to a threshold fluorescence based on the determined distance and the measured intensity of the fluorescence light at the third wavelength.

Alternatively, or additionally to any of the examples above, the first wavelength can have a first water absorption coefficient higher than a second water absorption coefficient of the second wavelength.

Alternatively, or additionally to any of the examples above, the ratio of the first water absorption coefficient to the second water absorption coefficient can be at least 2 to 1.

Alternatively, or additionally to any of the examples above, the wavelength can be approximately 200 nm to 700 nm.

Alternatively, or additionally to any of the examples above, the instructions, when executed by the processor, further cause the processor to compute a ratio of the intensity of the first wavelength to the intensity of the second wavelength. Determining the distance between the distal end of the optical fiber and the target can be based on the ratio of the first intensity to the second intensity.

Alternatively, or additionally to any of the examples above, the system includes a third laser source generating a therapeutic laser beam of a fifth wavelength. The instructions, when executed by the processor, further cause the processor to generate a therapeutic laser beam of the fifth wavelength from the third laser source and, based on determining that the fluorescence of the target is less than the threshold value, halt the therapeutic laser beam.

Alternatively, or additionally to any of the examples above, the system includes a switch and the processor generating the therapeutic laser beam is in response to the actuation of the switch. The instructions, when executed by the processor, further cause the processor to halt the therapeutic laser beam based on release of the switch.

Alternatively, or additionally to any of the examples above, the instructions, when executed by the processor, further cause the processor to compare the calculated distance against a threshold distance and halt the therapeutic laser beam based on determining that the calculated distance is greater than the threshold distance.

Alternatively, or additionally to any of the examples above, the second laser source is a fast-excitation laser source having activation and deactivation times of less than 10 μs.

Alternatively, or additionally to any of the examples above, the system includes a high-speed optical switch with a switching time of less than 1 μs.

Alternatively, or additionally to any of the examples above, the system includes an optical chopper configured to occlude the aiming beam for more than 99% of each ms during system operation.

In another example, a method includes the steps of determining a first intensity value based on first reflected laser light corresponding to laser light of a first wavelength, wherein the laser light of the first wavelength exits a distal end of an optical fiber and the first reflected laser light is reflected by a target and enters the distal end of the optical fiber; determining a second intensity value based on second reflected laser light corresponding to laser light of a second wavelength, wherein the laser light of the second wavelength exits the distal end of the optical fiber and the second reflected laser light is reflected by the target and enters the distal end of the optical fiber; determining a third intensity value corresponding fluoresced laser light corresponding to laser light of a fourth wavelength, wherein a laser light of a third wavelength exits the distal end of the optical fiber and the fluoresced laser light is excited from the target by the light of the third wavelength and enters the distal end of the optical fiber; computing a ratio of the first intensity value and the second intensity value; determining a distance between the distal end of the optical fiber and the target based on the computed ratio; and determining whether a calculated luminescence of the target is greater than a threshold luminescence based on the determined distance and the third intensity value.

Alternatively, or additionally to any of the examples above, the method further includes the step of halting a therapeutic laser light of a fifth wavelength in response to determining that the calculated luminescence of the target is less than the threshold luminescence.

Alternatively, or additionally to any of the examples above, the laser light of the third wavelength can be emitted for a duration of fewer than 10 μs each ms.

In another example, an endoscopic surgical system includes an LETD (light emitting transmitting and detecting) system with a first laser source to generate a first reference laser beam of a first wavelength and a second reference laser beam at a second wavelength, a second laser source to generate a fluorescence excitation laser beam of a third wavelength, a reference light detector to measure intensity of the reflected light at the first and second wavelengths, and a fluorescence light detector to measure intensity of light at a fourth wavelength fluoresced from a target in response to the emission of the fluorescence excitation laser beam. The endoscopic surgical system also includes an endoscopic probe comprising an optical fiber and an imager, an optical fiber having a distal end and a proximal end, the optical fiber configured to receive laser light from the first and second laser sources at the proximal end, to emit the laser light out of the distal end, and to receive reflected laser light into the distal end; and a processor and memory comprising instructions that when executed by the processor cause the processor to determine a distance from the distal end of the optical fiber to a target based on the measured intensity of the reflected light at the first and second wavelengths and determine whether a fluorescence of a target is greater than or equal to a threshold fluorescence based on the determined distance and the measured intensity of the fluorescence light at the fourth wavelength.

These and other features and advantages of the present disclosure will be readily apparent from the following detailed description, the scope of the claimed invention being set out in the appended claims.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates a system in accordance with at least one embodiment.

FIG. 2 illustrates a portion of the system of FIG. 1, in accordance with at least one embodiment.

FIGS. 3A and 3B illustrate an LETD, in accordance with at least one embodiment.

FIG. 4 is a flowchart depicting a process for differentiating stone and tissue, in accordance with at least one embodiment.

FIG. 5 is a flowchart depicting a process for automatically adjusting parameters of a medical laser console based on evaluating a target in accordance with at least one embodiment.

FIG. 6 is a flowchart depicting a process for automatically stopping lasing in accordance with at least one embodiment.

FIG. 7 is a flowchart depicting a process for automatically starting lasing in accordance with at least one embodiment.

FIG. 8 illustrates another system, in accordance with at least one embodiment.

FIG. 9 illustrates another system, in accordance with at least one embodiment.

FIG. 10 illustrates a computer-readable storage medium in accordance with at least one embodiment.

FIG. 11 illustrates a computing system in accordance with at least one embodiment.

### DETAILED DESCRIPTION

The present disclosure provides a method and system for distinguishing between a stone and a tissue based on reflected light from the stone or the tissue. It is to be appreciated that the efficiency of treatments using lasers often depend upon the relative position and orientation of the optical fiber tip with respect to the target. Further, the safety of the patient often depends on accurate aiming of the distal end of the optical fiber at the intended target. For example, where a stone is the intended target, unintentionally activating the laser while aimed at tissue could damage the tissue. This may lead to unnecessary complications, and in some cases, it can also lead to permanent damage to the tissue, which could make portions of the body of the subject dysfunctional. Discharge of therapeutic laser energy when not aimed at the target can have additional negative effects, including damaging one or more surgical instruments and/or unnecessarily increasing the temperature of the operative environment.

The method includes illuminating, by a light emitting transmitting and detecting (LETD) system, a target with laser light of different wavelengths having low-water and high-water absorption coefficients, using different laser light sources. The wavelengths may be selected in such a way that, they are close to each other and belong to the same “nm scale.” Further, the LETD system receives returned signals corresponding to the incident laser light of different wavelengths. The returned signals comprise light beams reflected from the target post illumination. The one or more light detectors configured in the LETD system may detect the returned signals to measure intensity values of the returned signals of a specific wavelength. Using the measured intensity values, a controller may determine a distance to the target.

The LETD system also illuminates the target with a fluorescence excitation beam, which is of a shorter wavelength at which there is much higher fluorescence excitation, and therefore detectible luminescence, for valid targets such as urinary stones than for the surrounding tissue. Based on the measured luminescence and the determined distance, the controller may determine whether the target is tissue or a stone.

The present disclosure uses the described LETD system in different configurations comprising different arrangements of various optical components, such as beam combiners, beam splitters, polarizers, collimators, wave division multiplexers (WDM), light detectors and the like. The present disclosure further enables accurate determination of whether the laser is incident on a stone or tissue and is compatible with different types of targets. Further, the present disclosure may be used for the purpose of controlling and/or adjusting one or more operational parameters. For instance, during a treatment, the target may move around, back, and forth or otherwise, or may change one or more of its shape, size, composition, pigment, and color. Therefore, parameters for the laser sources that are pre-set before initiating lasing on the target, may become less effective. Conventionally, such pre-set parameters are manually changed, which may be error prone and time consuming, or in some cases the pre-set parameters may be left unchanged which may lead to scenarios where the optical fiber may be too close or too far from the target. Therefore, the present disclosure allows automatic and real-time monitoring of the distance between the optical fiber end and the target, whether the laser light is incident on stone or tissue, and further enables automatically changing of the pre-set lasing parameters to adjust the lasing in accordance with the target shape, position etc. and to provide a higher likelihood of achieving the desired result or outcome from the treatment.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

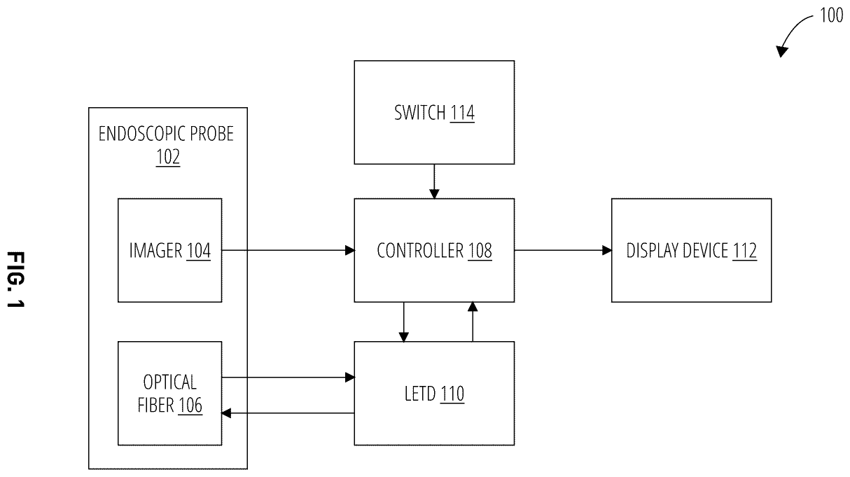

FIG. 1 shows an exemplary system 100 for estimating a distance between a fiber end and a target and/or distinguishing between tissue and stone, in accordance with some embodiments of the present disclosure. In some embodiments, the exemplary system 100 comprises an endoscopic probe 102 with an imager 104 and an optical fiber 106, a controller 108, a light emitting, transmitting, detecting (LETD) system 110, and a display device 112.

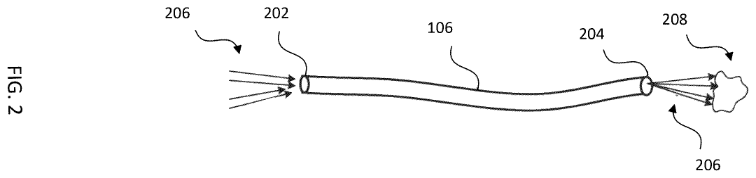

As depicted more fully in FIG. 2, the optical fiber 106 comprises a proximal end 202 and a distal end 204. The proximal end 202 is the end of the optical fiber 106 through which light beams 206 enter while the distal end 204 is the end of the optical fiber 106 through which light beams 206 are emitted and via which light beams 206 can be directed onto the target 208. For example, this figure depicts light beams 206 entering the optical fiber 106 at the proximal end 202, propagating through length of the optical fiber 106, exiting the optical fiber 106 at the distal end 204, and being incident on the target 208 from the distal end 204 of the optical fiber 106.

The light beams may be beams directed from a light source (e.g., included in LETD system 110, or the like). The light source can be a laser light source. As an example, such a laser light sources may include, but is not limited to, solid-state lasers, gas lasers, diode lasers, and fiber lasers. The light beams may include one or more of an aiming beam, a treatment beam, a fluorescence excitation beam, a distance measurement beam, and any other beam transmitted through the optical fiber 106.

In various embodiments, a fluorescence excitation beam may include a light beam of low intensity that is transmitted through the optical fiber 106 to excite target fluorescence radiation to estimate the target type. With some embodiments, the fluorescence excitation beam can be a laser beam having a wavelength in the visible range. As a specific example, the fluorescence excitation beam can be a “green” light laser, such as, a laser emitting light having a wavelength of 520 nm, 500 nm to 580 nm, or the like. In such embodiments, the light from the fluorescence excitation beam stimulates significantly lower fluorescence in the soft tissue (e.g., at least in part due to differences in soft tissue chemical compositions and molecular structures vs urinary stones). As such, the luminescence of soft tissue will be significantly lower than that of a stone.

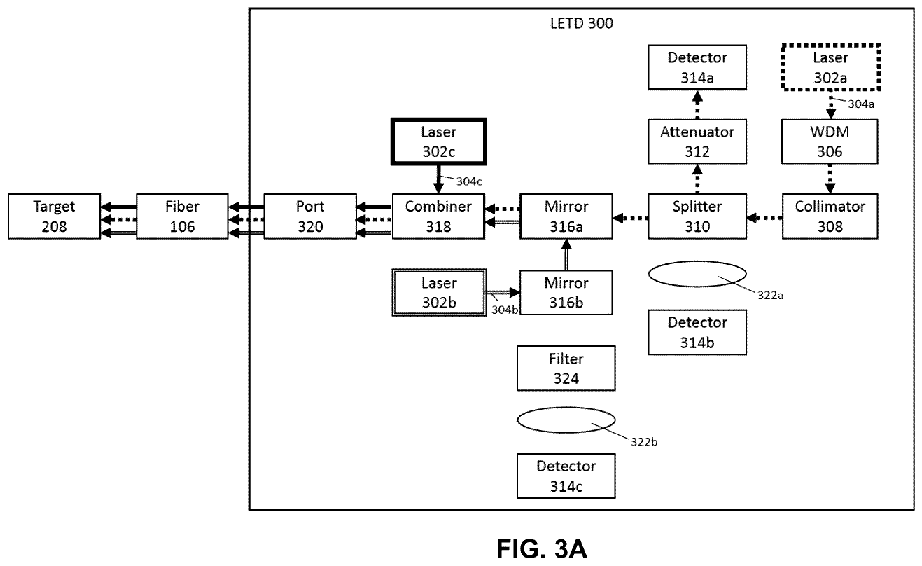

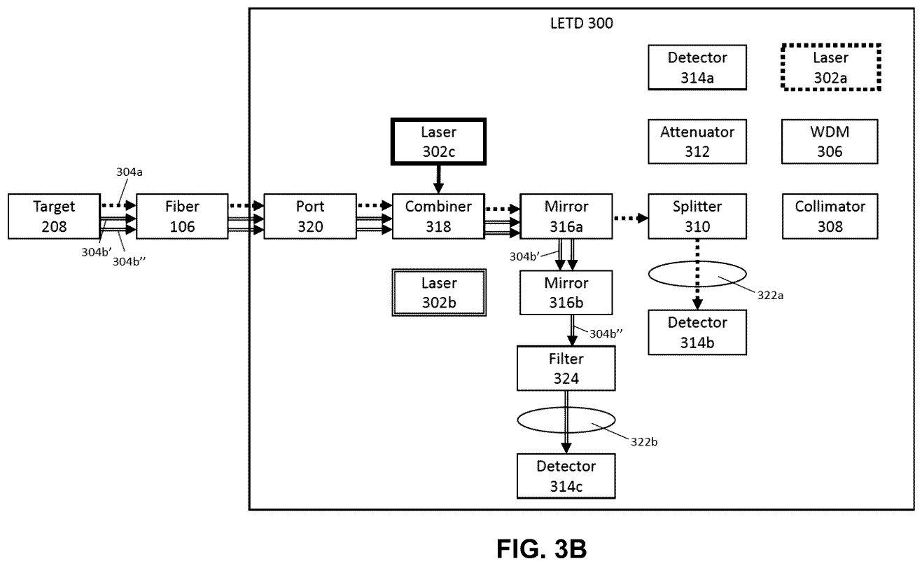

In several embodiments, a treatment beam may include a light beam of high intensity that is transmitted through the optical fiber 106 to treat the target 208. In some embodiments, the different light beams may be produced by one or more laser light sources. As a specific example, the fluorescence excitation beam may be generated by one laser source and the treatment beam may be generated by another laser source. In another example, both the fluorescence excitation beam and the treatment beam may be generated by a single laser source. With yet another example, different laser light sources may be used to generate light beams of different wavelengths, characteristics, and the like. This is described in greater detail below, for example, with respect to FIG. 3.

The optical fiber 106 may be in optical communication with the LETD system 110 as shown in the FIG. 1 and arranged to receive light beams, to be aimed at the target 206, and to deliver reflected light beams that reflect from the surface of and region around the target 208. In some embodiments, the optical fiber 106 may be optically, mechanically, and/or electrically coupled with the LETD system 110 via a port (shown in other figures herein).

In some embodiments, the LETD system 110 comprises optical components which may include, but are not limited to, one or more of laser light sources, polarizers, beam splitters, beam combiners, light detector, wavelength division multiplexers, collimators, circulators, that are configured in various combinations, as explained in detail in further parts of the present disclosure.

In many embodiments, laser light sources are configured to generate laser light beams, such as a low intensity aiming beam for the purpose of aiming the light beams 206 at the target 206 and a high intensity treatment beam for treating the target 206, and/or light beams with varying characteristics (e.g., intensities, wavelengths, etcetera) based on the application. Each laser light source may be configured to generate laser light having different wavelengths, where each of the different wavelengths can have different water absorption coefficients. Further, each laser light source may have the same aperture or different apertures. In some embodiments, each laser light source may be designated with a different purpose, for instance, one laser light source may be configured to generate aiming beams and/or fluorescence excitation beams of a particular intensity and one laser light source may be configured to generate a treatment beam of a particular intensity, and one or more laser light sources may be configured to generate light beams of a specific wavelength having specific water absorption coefficient. Additionally, each laser light source may be configured to generate polarized laser light or unpolarized/depolarized light.

Polarizers may include the optical components that act as an optical filter. For example, polarizers may be configured to allow light beams of a specific polarization to pass through, and to block the light beams of different polarizations. Therefore, when undefined light (or light beams of mixed polarity) is provided as input to a polarizer, the polarizer provides a well-defined single polarized light beam as an output.

Beam splitters may include the optical components used to split incident light at a designated ratio into two separate beams. Further, beam splitters may be arranged to manipulate light to be incident at a desired angle of incidence (AOI). Therefore, in many embodiments, a beam splitter can be primarily configured with two parameters, a ratio of separation and an AOI. The ratio of separation comprises the ratio of reflection to transmission (reflection/transmission (R/T) ratio) of the beam splitter. Accordingly, as used herein, if the ratio of separation for a beam splitter is indicated as 50:50, it means that the beam splitter splits the incident light beams in a R/T ratio of 50:50. In other words, the beam splitter splits the incident light beams by changing the incident light by reflecting 50 percent and transmitting the other 50 percent. Further, as an example, if the AOI for the beam splitter is indicated as 45 degrees, it means that the beam splitter ensures that the light beams would be incident at an angle of 45 degrees. Beam splitters may include, but are not limited to, polarizing beam splitters and non-polarizing beam splitters. Polarizing beam splitters may split incident light based on the S-polarization component and P-polarization component, such as, for example by reflecting the S-polarized component of light and transmitting the P-polarized component of light (or vice-versa). In some embodiments, non-polarizing beam splitters may split incident light beams based on a specific R/T ratio while maintaining the original polarization state of the incident light beams.

Beam combiners may include partial reflectors that combine two or more wavelengths of light, such as by using the principle of transmission and reflection as explained above. In many embodiments, a beam combiner may be a combination of beam splitters and mirrors, which perform the functionality of combining light of two or more wavelengths.

Light detectors may include devices that detect and/or measure characteristics of light beams and encode the detected and/or measured characteristics in electrical signals. For example, light detectors may detect the specific type of light beams (as preconfigured), and convert the light energy associated with the detected light beams into electrical signals.

A collimator may include a device that narrows down light beams. To narrow down the light beam, a collimator may be configured to cause the directions of motion to become more aligned in a specific direction (for example, parallel rays), or to cause the spatial cross section of the beam to become smaller. In many embodiments, a collimator may be used to change diverging light from a point source into a parallel beam.

A circulator may include a multi-port optical device configured to receive and emit light via a predetermined sequence of the multiple ports. For example, a circulator may include a three (or four, or five, etc.) port optical device designed such that, light entering any one port exits from the next port. In one such example, light entering a first port may exit a second port, light entering the second port may exit a third port, and light entering the third port may exit the first port. Oftentimes circulators may be utilized to allow light beams to travel in only one direction.

It is noted that where optical component described herein list specific parameters, such as, a beam splitter having an R/T ratio of 50:50 and an AOI of 45 degrees, these parameters are provided for general understanding of the concepts disclosed and not to be limiting. As a specific example, a beam splitter could be provided in various embodiments described herein having a different R/T ratio and/or AOI than specified here without departing from the scope of the disclosure and claims. In one such example, an AOI of 40 degrees may be utilized. In another such example an R/T ratio of 47:53 may be utilized.

The LETD system 110 is further associated with a controller 108 and/or a communication network (not shown). In some embodiments, the communication network may be a wired communication network or a wireless communication network. The controller 108 may be configured to receive measured values from the LETD system 110 and estimate the distance between the distal end 204 of the optical fiber 106 and the target 208 based on the measured values. The controller 108 may be configured to receive measured values from the LETD system 110 and estimate the type of the target 208 based on the measured values. In some embodiments, the controller 108 may be a standalone device with the processing capability required for distance estimation and target type estimation. For example, controller 108 can include circuitry arranged to determine a distance and target type based on electrical signals received from the LETD system 110. As another example, controller 108 can include circuitry and memory comprising instructions, which when executed by the circuitry cause the circuitry to determine a distance and target type based on electrical signals received from the LETD system 110. Still, in some other embodiments, the controller 108 may be a computing device such as a laptop, a desktop, a mobile phone, a tablet phone, and the like, configured to perform the distance and target type estimation using their processing capability.

The controller 108 further receives signals from any number of user interface devices, represented by a switch 112. The switch 112 may be any mechanical or electrical interface such as button, pedal, toggle, or the like. In some implementations, the switch 112 may be a foot pedal used by an operator of the device 100 to discharge the therapeutic laser. As described herein, multiple pieces of information may be processed by the controller 108 in determining whether and when to energize a therapeutic laser source; whether or not the switch 112 is actuated may be one such signal.

Various exemplary configurations for estimating distance between the fiber end and the target and/or distinguishing between stone and tissue are explained in detail below. However, values and parameters associated with different optical components used in each of the below explained configurations, should be considered purely exemplary, and not be construed as a limitation of the present disclosure.

FIGS. 3A and 3B illustrate a LETD system 300, which can be implemented as the LETD system 110 of system 100. The LETD system 300 can be configured to estimate distance between a fiber end and a target and/or distinguish between stone or tissue, in accordance with some embodiments of the present disclosure. As depicted, LETD system 300 includes three laser sources: a reference laser source 302a, a fluorescence excitation laser source 302b, and a therapeutic laser source 302c. With some embodiments, the laser sources 302a and 302b can be polarized laser sources. In some implementations, multiple beams as described herein may be produced by the same laser source; for example, the reference laser source may also produce the fluorescence excitation beam, or the therapeutic laser source may also produce the fluorescence excitation beam.

The various components of the LETD system 300, and most particularly the laser sources 302a-c, are controlled by the controller 108, which uses the methods described herein to determine when each laser source will discharge, for how long, and at what intensities and frequencies.

As shown in FIG. 3A, a beam 304a emitted by the reference laser source 302a is received at a wave division multiplexing coupler 306 and passes through a collimator 308. The beam 304a is divided at a splitter 310; a first branch is processed by an attenuator 312 and then into a reference detector 314a. The other split branch of the beam 304a is transmitted through a first dichroic mirror 316a, into a beam combiner 318, and through a port 320 to enter the fiber 106 and hit the target 208. Light 304a of the same wavelength as the reference laser beam 304a returns from the target as shown in FIG. 3B, passing back through the fiber 106, port 320, combiner 318, dichroic mirror 316a and splitter 310. The beam 304a is then directed by a focusing lens 322a into a return signal detector 314b. The relative intensity measured between the reference detector 314a and return signal detector 314b is used in calculating the distance between the fiber 106 and the target 208 as further described herein.

The reference laser source 302a may be one or more devices capable of emitting multiple frequencies of light in order to measure relative intensities as described herein. For example, the laser source 302a could produce a first frequency at 1340 nm representing a frequency having higher water absorption and a second frequency at 1310 nm, having lower water absorption. The two are labelled as “HI” and “LO” respectively in the equations below.

Returning to FIG. 3A, a beam 304b emitted by the fluorescence excitation laser source 302b is reflected by two dichroic mirrors 318b and 316a before following the same path as the reference beam 304a: into the combiner 318, out the port 320, and through the fiber 106 to hit the target 208.

Light from the excitation laser beam 304b may at least partially reflect from the target 208 while also exciting fluorescence in a second shifted wavelength, so that both light frequencies enter the fiber 106, shown in FIG. 3B as a reflectance beam 304b′ and a fluorescence beam 304b″. Both beams 304b′ and 304b″ pass back through the fiber 106, port 320, combiner 318, and dichroic mirror 316a. Unlike the reference beam 304a, the reflectance and fluorescence beams 304b′ and 304b″ are deflected to a second dichroic mirror 316b which is selected to distinguish between them. The second dichroic mirror 316b partially blocks the reflectance wavelength of beam 304b′ (the same wavelength as excitation laser beam 304b) and passes the fluorescence wavelength of beam 304b″. A long pass filter 324 may be employed along the return path of the returned beams 304b′ and 304b″ to ensure that only the fluorescence wavelength will be directed by a focusing lens 322b into a fluorescence beam detector 314c.

A therapeutic laser source 302c emits a beam 304c that passes through the combiner 318, port 320, and fiber 106 to be directed at the target 208 for ablation, fragmentation, or the like. When and whether the therapeutic laser source 302c is activated depends on evaluation of the beams received by the detectors 314a-314c as further explained.

In some implementations, the relative intensity of the signal from the detector 314a and the detector 314b may be used to determine the distance between the fiber 106 and target 208. Controller 108 may estimate a distance between the distal end 204 of the optical fiber 106 and the target 208 based on the measured intensities of the returned signals. In some embodiments, the controller 108 may substitute the measured intensities in the Equation 1 as shown below:

$$Intensity of the returned signal = R ⋆ {e}^{( - λ ⋆ X )} \qquad Equation 1$$

In the above Equation 1, “R” refers to target reflection coefficient, which is affected by target composition, target color/pigment, target angle, target surface and the like; “λ” refers to water absorption coefficient of a specific wavelength; and “X” refers to distance between the distal end 204 of the optical fiber 106 and the target 208.

In the above Equation 1, “X” and “R” are unknown parameters which need to be determined by the controller 108. Therefore, to determine the values of “X” and “R”, the controller 108 may substitute the two measured intensity values in the above Equation 1, thereby obtaining two equations with substituted values of measured intensity and the water absorption coefficient of the corresponding wavelength. For instance, the two equations with substituted values may be as shown below.

$${I}_{( H I )} = R ⋆ {e}^{( - λ\_ HI ⋆ X )} \qquad Equation 1.1$$

$${I}_{( L O )} = R ⋆ {e}^{( - λ\_ LO ⋆ X )} \qquad Equation 1.2$$

The controller 108 may further simplify the above substituted Equations 1.1 and 1.2 as follows: compute ratio of measured intensity values obtained for the returned signal of two different wavelengths using Equation 2.1; and determine distance value using the natural logarithm as shown in Equation 2.2.

$$\frac{{I}_{( HI )}}{{I}_{( LO )}} = \frac{R}{R} ⋆ {e}^{( - {λ}_{LO} - {λ}_{HI} ) ⋆ X} \qquad Equation 2.1$$

$$X = \frac{ln ( \frac{{I}_{( HI )}}{{I}_{( LO )}} )}{{λ}_{LO} - {λ}_{HI}} \qquad Equation 2.2$$

Therefore, the controller 108 may estimate the distance X between the distal end 204 of the optical fiber 106 and the target 208, by simplifying Equations 1.1 and 1.2 as shown above. In the above Equation 2.2, “ln” refers to natural logarithm. In some embodiments, the distance X may be measured in millimeters. In some embodiments, X is the same distance for both wavelengths and R (target reflection) is almost identical for both wavelengths when the selected wavelengths are close to each other on the “nm scale”. In some embodiments, wavelengths may be considered close to each other on the “nm scale” when they are within 250 nm (e.g., 1310 nm and 1340 nm or 1310 nm and 1550 m). However, in many embodiments, wavelengths with closer R values may be selected. Accordingly, 1310 nm and 1340 nm may be selected over 1310 nm and 1550 nm. With some examples of the present disclosure, the two wavelengths of reference laser source 302a can be arranged to emit light having wavelengths that are within 100 nm of each other.

The above calculated distance X may be considered when evaluating the fluorescence of the target 208 using the fluorescence excitation beam 304b. In some implementations, the measured fluorescence intensity may be corrected by a distance factor as described in Equation 3:

$$Distance factor = \frac{1}{{( 1 + \frac{4 \cdot X \cdot N A}{d} )}^{2}} \qquad Equation 3$$

In this equation, NA is the effective numerical aperture of the laser fiber 106 and d is the fiber core diameter. When the distance-corrected fluorescence intensity measured by the detector 314c exceeds a threshold value, the target 208 is determined to be stone and the therapeutic laser 302c can be used. When the intensity is below the threshold value, the target 208 is determined to be an improper target for laser operation, such as patient tissue.

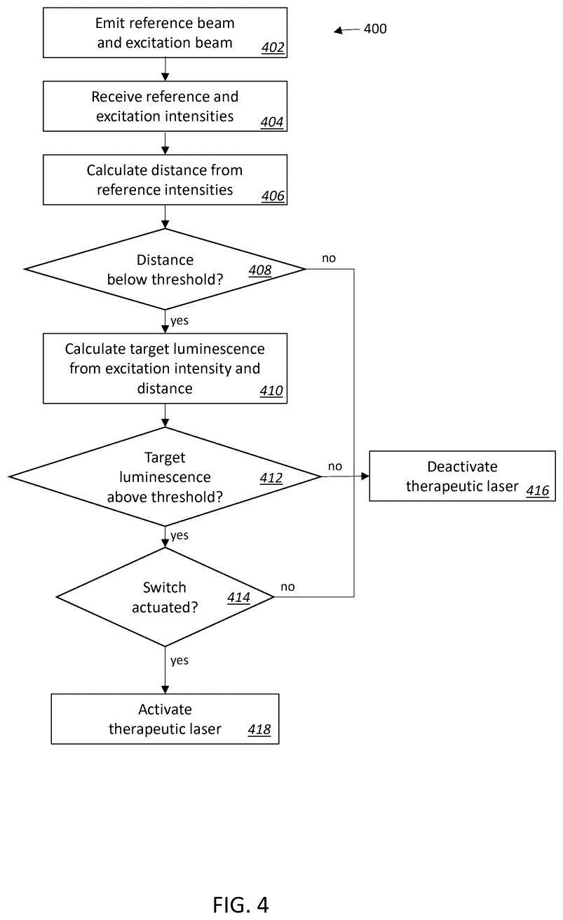

A flowchart 400 is shown in FIG. 4 illustrating an exemplary process for evaluating a target in accordance with the present disclosure. The steps as labeled should be read broadly; for example, the deactivation step 416 would also include not energizing the laser source if it were not yet activated. Similarly, the activation step 418 would also include maintaining the beam if it were already activated.

The laser sources emit reference and fluorescence excitation beams (step 402), and the light reflected off the target from those beams are measured by the detectors (step 404). The intensities of the reference beams are used to calculate a distance between the laser fiber tip and the target (step 406).

If the calculated distance is below a certain threshold, (yes branch of decision step 408), then the distance value is used in conjunction with the intensity of the detected fluorescence frequency to calculate a luminescence value for the target (step 410). The distance threshold may be dependent on factors such as an identified target type, the imaging capabilities of the endoscope, and the parameters of the procedure. If the calculated distance is farther then the threshold (no branch of decision step 408), then the controller 108 will not activate the therapeutic laser source or will deactivate that source (step 416).

In some implementations, the controller 108 may also take the calculated distance into account when determining the intensity of the therapeutic beam 304c. For example, if tip of the fiber 106 is calculated as being in contact with the target 208, the therapeutic laser source 302c may be activated at full power, while if the tip of the fiber 106 is calculated as being a significant distance from the target 208, the laser source 302c may be activated at only partial power. These values may be preset based on the target and procedure type but may also be set by the operator of the device 100.

At decision step 412, the system evaluates the calculated luminescence of the target against an established threshold. This threshold value may be itself determined by one or more factors, such as calibration data for the device and measured conditions within the patient. If the luminescence of the target is determined to be consistent with a kidney stone or other valid target (yes branch at decision step 412) and the switch is pressed or otherwise actuated (yes branch at decision step 412), then a therapeutic laser is activated or maintained (step 418). If either of these conditions are not met, such that either the adjusted luminescence falls below the threshold (no branch at decision step 412) or the switch is in an off position (no branch at decision step 414), then the laser source fails to activate or is cut off if already activated (step 416).

In some implementations, when the luminescence value is inconsistent with a valid target, but the switch is activated, then an indication may appear to the operator, such as through the display 112. In some implementations, the processor 108 may lock or otherwise restrict the ability of the therapeutic laser to activate until the measured luminescence exceeds the threshold. In other implementations, while one or more warning indications or safety alerts may be provided to the operator, the system may still permit deployment of the therapeutic laser based on the operator's judgment.

The initial intensity of the fluorescence excitation beam 304b relates directly to the signal strength of the resulting luminescence measurement for the target. Unfortunately, because the fluorescence excitation beam 304b may be visible light, it may negatively affect the operator's ability to observe the fiber and stone on the imager 104. To address this, the fluorescence excitation beam 304b may be pulsed intermittently and be at a very low intensity in average (the human eye and scope camera detects to an average intensity)

In some implementations, the laser source 302b may be capable of fast excitation, so that the source itself can be activated and deactivated within a set duration such as 10 μs or likewise. An intense pulse of more than 5 mW may be used with a low duty cycle; for example, the source 302b may be excited for 10 μs and deactivated for 1 ms for a duty cycle of less than 1%.

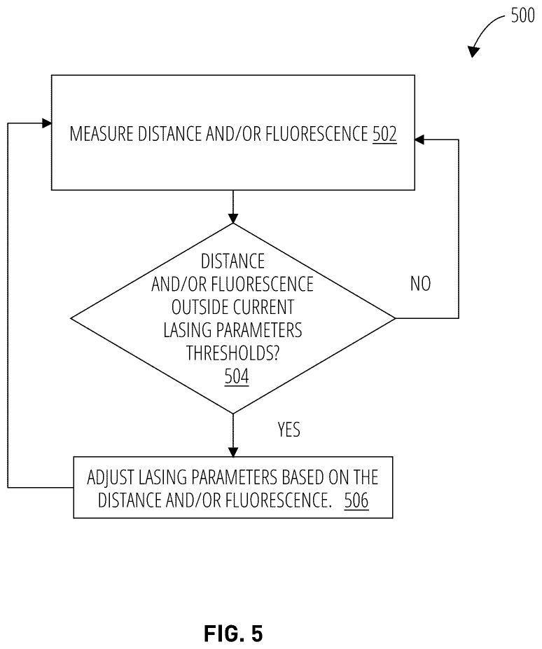

A flowchart 500 is shown in FIG. 5 illustrating an exemplary process for automatically adjusting parameters of a medical laser console based on evaluating a target in accordance with the present disclosure. In some examples, the process shown in flowchart 500 can correspond to operations implemented by controller 108 at step 416 and/or 418. For example, 108 can implement logic from flowchart 500 to carry out the deactivation and activation steps of 416 and 418.

Flowchart 500 can include a step 502 to determine, or measure, the distance and/or fluorescence as described herein. For example, controller 108 can implement portions of flowchart 400 to determine the distance and/or fluorescence as discussed herein.

Flowchart 500 can further include step 504 to determine whether to automatically adjust lasing parameters (e.g., power, repetition rate, pulse width, etc.) based on the determined distance and fluorescence (e.g., as determined in flowchart 400, or the like) and if adjustment of the lasing parameters is necessitated (e.g., based on the distance and/or fluorescence) to adjust the lasing parameters (e.g., at step 506). Conversely, where the laser parameters do not need to be adjusted, the flowchart 500 can return to step 502. That is. The flowchart 500 is intended to be performed iteratively during a medical procedure.

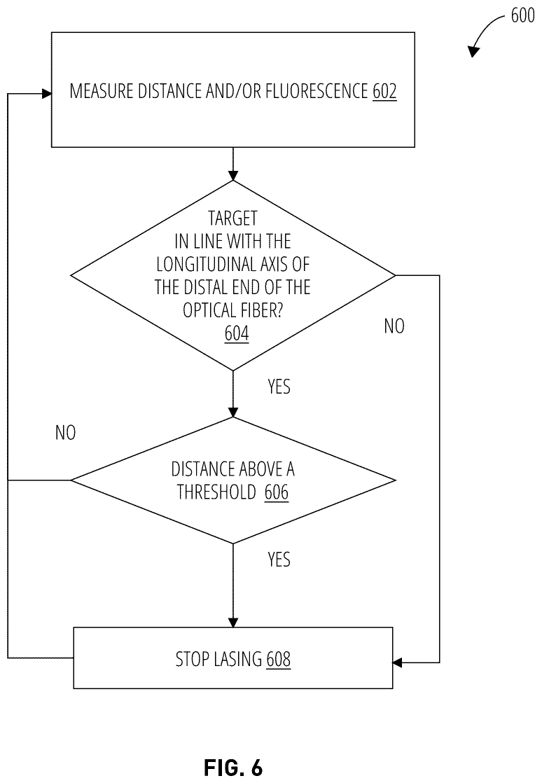

A flowchart 600 is shown in FIG. 6 illustrating an exemplary process for automatically stopping lasing based on evaluating a target in accordance with the present disclosure. In some examples, the process shown in flowchart 600 can correspond to operations implemented by controller 108 at step 416 to deactivate lasing by the medical laser console.

Flowchart 600 can include a step 602 to determine, or measure, the distance and/or fluorescence as described herein. For example, controller 108 can implement portions of flowchart 400 to determine the distance and/or fluorescence as discussed herein.

Flowchart 600 can further include step 604 to determine whether the target 208 is in line with the longitudinal axis of the distal end 204 of the optical fiber 106. Or said differently, controller 108 can determine whether the target 208 is “in front” off the distal end 204 of the optical fiber 106.

Where the target is in line with the longitudinal axis of the distal end 204 of the optical fiber 106, the flowchart continues to step 606 where a determination is made as to whether the distance is above a threshold (e.g., like step 408).

Where the distance is above the threshold or where target is not in line with the distal end of the optical fiber, the controller 108 can deactivate the laser (e.g., step 416).

Conversely, where the target is in line with the longitudinal axis of the distal end of the optical fiber and the distance is not above the threshold, the flowchart 600 can return to step 602. That is. The flowchart 600 is intended to be performed iteratively during a medical procedure.

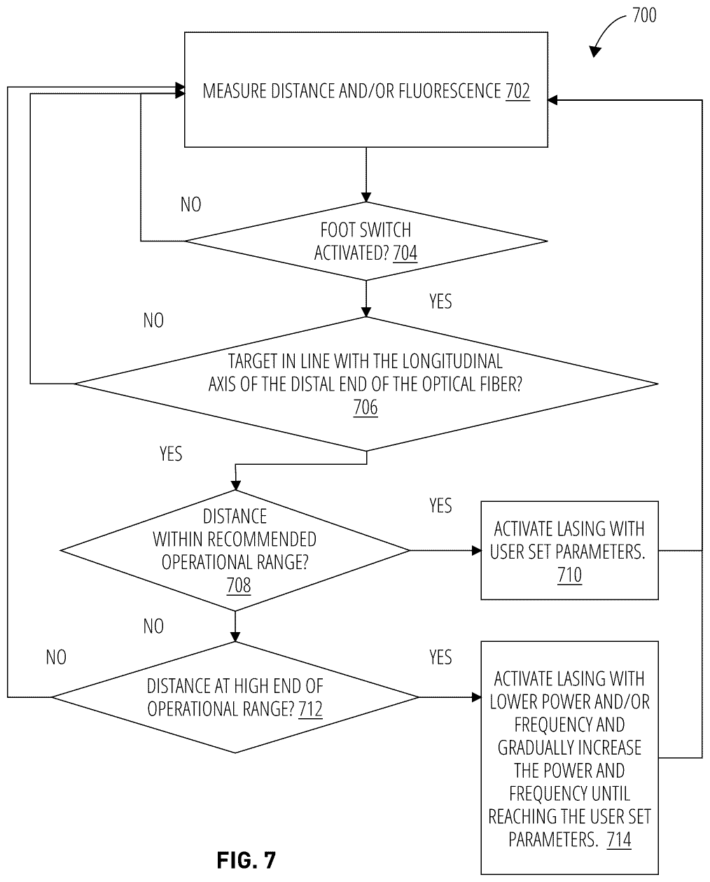

A flowchart 700 is shown in FIG. 7 illustrating an exemplary process for automatically starting lasing based on evaluating a target in accordance with the present disclosure. In some examples, the process shown in flowchart 700 can correspond to operations implemented by controller 108 at step 418 to activate lasing by the medical laser console.

Flowchart 700 can include a step 702 to determine, or measure, the distance and/or fluorescence as described herein. For example, controller 108 can implement portions of flowchart 400 to determine the distance and/or fluorescence as discussed herein.

Flowchart 700 can further include step 704 to determine whether a foot switch (e.g., switch 112, or the like) is active. Where the foot switch is active, the flowchart 700 can continue to step 706 and determine whether the target 208 is in line with the longitudinal axis of the distal end 204 of the optical fiber 106. Or said differently, controller 108 can determine whether the target 208 is “in front” off the distal end 204 of the optical fiber 106.

Where the foot switch is not active or the target is not “in front” of the distal end of the optical fiber, the flowchart 700 can return to step 702. That is. The flowchart 600 is intended to be performed iteratively during a medical procedure.

Where the foot switch is active and the target is “in front” of the distal end of the optical fiber, the flowchart 700 can include step 708 to determine whether the distance is within a recommended operational range (e.g., 0 to 1 mm, or the like). Where the distance is within the recommended operation range, flowchart 700 can continue to step 710 to activate lasing with the user established or user set parameters (e.g., power, frequency, etc.)

Where the distance is not within the recommended operational range, the flowchart 700 can continue to step 712 to determine whether the distance within an “upper end” of the operational range (e.g., 1 to 2 mm, or the like). Where the distance is within the upper end of the operation range, flowchart 700 can continue to step 714 to activate lasing with lower power and/or lower frequency than defined by the established user parameters to prevent retropulsion. The controller can then gradually increase the power and/or frequency up to the established user defined parameters as the distal end 204 of the optical fiber 106 approaches the target 208.

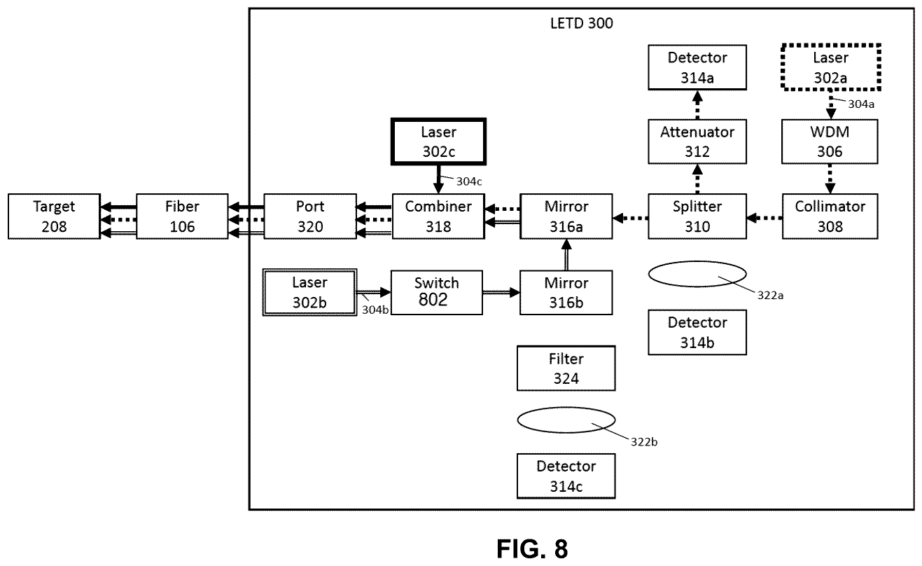

As shown in FIG. 8, an optical high-speed switch 802 may be added to the LETD 300 between the laser source 302b and the dichroic mirror 318b. In this embodiment, the laser source 302b itself may stay excited, but the switch is operated with an on time of less than 10 μs per ms. To adequately control the pulse cycle, the switch 502 can have an activation time of less than 1 μs.

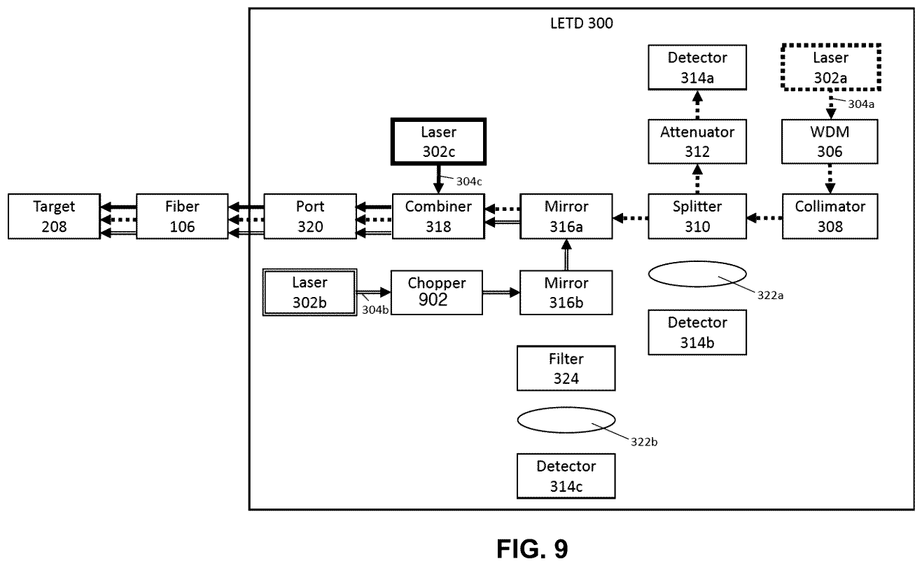

As shown in FIG. 9, an optical chopper 902 may be added to the LETD instead of the high-speed switch. The optical chopper may be, for example, a linear chopper or a wheel chopper as known in the art. The chopper may be configured to occlude the beam 99% of the time so that the beam passes to the mirror 318b for only 10 μs once every ms.

In some implementations, the fluorescence excitation beam duty cycle may be synchronized with the imager 104 of the endoscope probe 102. For example, where the imager 104 is a camera with a framerate of 60 frames per second, the processor 108 may control the LETD to set aside a window of 1 ms each 16.667 ms in which no pulse is received. In other implementations, the fluorescence excitation beam duty cycle may be independent of the imager timing, but the low duty cycle may reduce the presence of the fluorescence excitation beam in the images to an acceptable level.

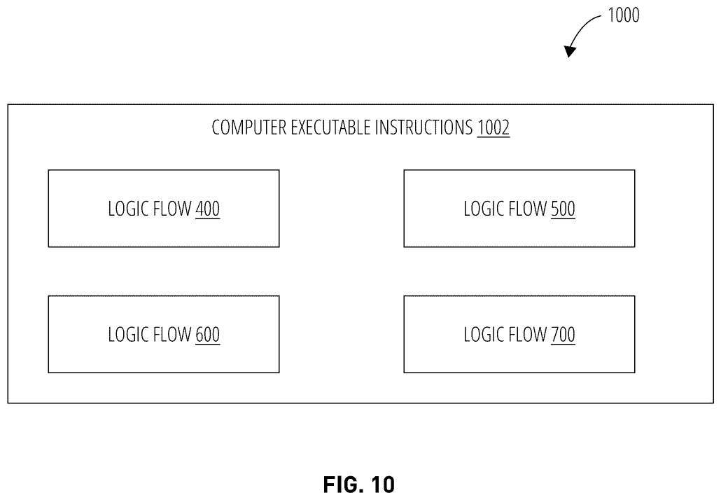

FIG. 10 illustrates computer-readable storage medium 1000. Computer-readable storage medium 1000 may comprise any non-transitory computer-readable storage medium or machine-readable storage medium, such as an optical, magnetic or semiconductor storage medium. In various embodiments, computer-readable storage medium 1000 may comprise an article of manufacture. In some embodiments, computer-readable storage medium 1000 may store computer executable instructions 1002 with which circuitry (e.g., controller 108, or the like) can execute. For example, computer executable instructions 1002 can include instructions to implement operations described with respect to a logic flow for flowchart 400, a logic flow for flowchart 500, a logic flow for flowchart 600, and/or a logic flow for flowchart 700. Examples of computer-readable storage medium 1000 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1002 may include any suitable type of code, such as source code, compiled code, interpreted code, executable code, static code, dynamic code, object-oriented code, visual code, and the like.

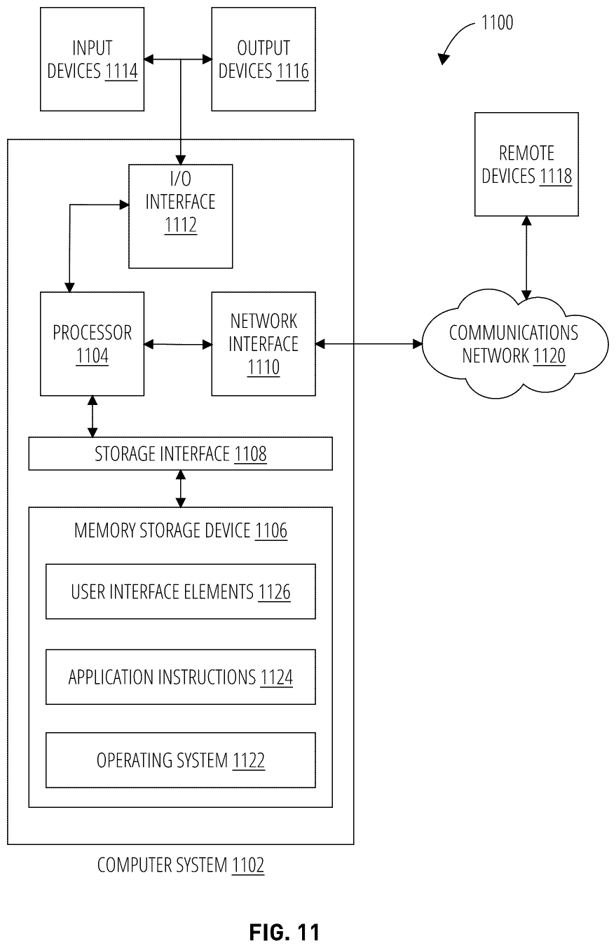

FIG. 11 is a block diagram of a computing environment 1100 including a computer system 1102 for implementing embodiments consistent with the present disclosure. In some embodiments, the computing environment 1100, or portion thereof (e.g., the computer system 1102) may comprise or be comprised in a laser system (e.g., the system 100, LETD 300, controller 108, etc.). Accordingly, in various embodiments, computer system 1102 may be used to determine a distance and/or fluorescence and automatically adjust lasing parameters and/or start or stop lasing based on the distance and/or fluorescence as described above.

The computer system 1102 may include a central processing unit (“CPU” or “processor”) 1104. The processor 1104 may include at least one data processor for executing instructions and/or program components for executing user or system-generated processes. A user may include a person, a person using a device such as those included in this disclosure, or another device. The processor 1104 may include specialized processing units such as integrated system (bus) controllers, memory management control units, floating point units, graphics processing units, neural processing units, digital signal processing units, etc. The processor 1104 may be disposed in communication with input devices 1114 and output devices 1116 via I/O interface 1112. The I/O interface 1112 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMAX, or the like), etc.

Using the I/O interface 1112, computer system 1102 may communicate with input devices 1114 and output devices 1116. In some embodiments, the processor 1104 may be disposed in communication with a communications network 1120 via a network interface 1110. In various embodiments, the communications network 1120 may be utilized to communicate with a remote memory storage device 1106, such as for accessing look-up tables, performing updates, or utilizing external resources. The network interface 1110 may communicate with the communications network 1120. The network interface 1110 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 1120 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 1120 may include a variety of network devices, including routers, bridges, servers, computing devices, storage devices, etcetera. In some embodiments, the processor 1104 may be disposed in communication with a memory storage device 1106 via a storage interface 1108. The storage interface 1108 may connect to memory storage device 1106 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 1106 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 1106 may store a collection of program or database components, including, without limitation, an operating system 1122, an application instructions 1124, and a user interface elements 1126. In various embodiments, the operating system 1122 may facilitate resource management and operation of the computer system 1102. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD® OPENBSD, etc.), LINUX® DISTRIBUTIONS (E.G., RED HAT®, UBUNTU®, KUBUNTU®, etc.), IBM® OS/2®, MICROSOFT® WINDOWS® (XP®, VISTA®/7/8, 10 etc.), APPLE® IOS®, GOOGLE™ ANDROID™, BLACKBERRY® OS, or the like.

The application instructions 1124 may include instructions that when executed by the processor 1104 cause the processor 1104 to perform one or more techniques, steps, procedures, and/or methods described herein, such to irrigate a site and irradiate a site as outlined herein. For example, application instructions 1124, when executed by processor 1104 can cause processor 1104 to perform the method 400, logic flow 500, method 800, and/or method 900.

The user interface elements 1126 may facilitate display, execution, interaction, manipulation, or operation of program components through textual or graphical facilities. For example, user interfaces may provide computer interaction interface elements on a display system operatively connected to the computer system 1102, such as cursors, icons, checkboxes, menus, scrollers, windows, widgets, etcetera. The user interface elements 1126 may be employed by application instructions 1124 and/or operating system 1122 to provide, for example, a user interface with which a user can interact with computer system 1102. In some embodiments, the user interface elements 1126 may be integrated with the display (not shown).

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. A system, comprising:
a first laser source to generate a first reference laser beam of a first wavelength and a second reference laser beam at a second wavelength;
a second laser source to generate a fluorescence excitation laser beam of a third wavelength;
an optical fiber having a distal end and a proximal end, the optical fiber configured to receive laser light from the first and second laser sources at the proximal end, to emit the laser light out of the distal end, and to receive reflected laser light into the distal end;
a reference light detector to measure intensity of the reflected light at the first and second wavelengths;
a fluorescence light detector to measure intensity of light at a fourth wavelength fluoresced from a target in response to the emission of the fluorescence excitation laser beam; and
a processor and memory comprising instructions that when executed by the processor cause the processor to:
determine a distance from the distal end of the optical fiber to the target based on the measured intensity of the reflected light at the first and second wavelengths; and
determine whether a fluorescence of a target is greater than or equal to a threshold fluorescence based on the determined distance and the measured intensity of the fluorescence light at the fourth wavelength.

2. The system of claim 1, wherein the first wavelength has a first water absorption coefficient higher than a second water absorption coefficient of the second wavelength.

3. The system of claim 2, wherein the ratio of the first water absorption coefficient to the second water absorption coefficient is at least 2 to 1.

4. The system of claim 1, wherein the third wavelength is approximately 200 nm to 700 nm.

5. The system of claim 1, wherein the instructions, when executed by the processor, further cause the processor to compute a ratio of the intensity of the first wavelength to the intensity of the second wavelength,
and wherein determining the distance between the distal end of the optical fiber and the target is based on the ratio of the first intensity to the second intensity.

6. The system of claim 1, further comprising a third laser source generating a therapeutic laser beam of a fifth wavelength;
wherein the instructions, when executed by the processor, further cause the processor to:
generate a therapeutic laser beam of the fifth wavelength from the third laser source, and
based on determining that the fluorescence of the target is less than the threshold value, halt the therapeutic laser beam.

7. The system of claim 6, further comprising a switch;
wherein the processor generating the therapeutic laser beam is in response to the actuation of the switch, and
wherein the instructions, when executed by the processor, further cause the processor to halt the therapeutic laser beam based on release of the switch.

8. The system of claim 6, wherein the instructions, when executed by the processor, further cause the processor to:
compare the calculated distance against a threshold distance, and
halt the therapeutic laser beam based on determining that the calculated distance is greater than the threshold distance.

9. The system of claim 1,
wherein the second laser source has an emission intensity of greater than 5 mW,
and wherein the instructions, when executed by the processor, further cause the optical fiber to receive the fluorescence excitation laser beam for less than 10 μs per ms of operation.

10. The system of claim 9, wherein the second laser source is a fast-excitation laser source having activation and deactivation times of less than 10 μs.

11. The system of 9, further comprising a high-speed optical switch with a switching time of less than 1 μs.

12. The system of claim 9, further comprising an optical chopper configured to occlude the aiming beam for more than 99% of each ms during system operation.

13. An endoscopic surgical system, comprising:
an LETD system, comprising:
a first laser source to generate a first reference laser beam of a first wavelength and a second reference laser beam at a second wavelength;
a second laser source to generate a fluorescence excitation laser beam of a third wavelength;
a reference light detector to measure intensity of the reflected light at the first and second wavelengths;
a fluorescence light detector to measure intensity of light at a fourth wavelength fluoresced from a target in response to the emission of the fluorescence excitation laser beam; and
an endoscopic probe comprising an optical fiber and an imager, an optical fiber having a distal end and a proximal end, the optical fiber configured to receive laser light from the first and second laser sources at the proximal end, to emit the laser light out of the distal end, and to receive reflected laser light into the distal end; and
a processor and memory comprising instructions that when executed by the processor cause the processor to:
determine a distance from the distal end of the optical fiber to a target based on the measured intensity of the reflected light at the first and second wavelengths; and
determine whether a fluorescence of a target is greater than or equal to a threshold fluorescence based on the determined distance and the measured intensity of the fluorescence light at the fourth wavelength.

14. The system of claim 13,
wherein the second laser source has an emission intensity of greater than 5 mW,
and wherein the instructions, when executed by the processor, further cause the optical fiber to receive the aiming laser beam for less than 10 μs per ms of operation.

15. The system of claim 9, wherein the second laser source is a fast-excitation laser source having activation and deactivation times of less than 10 μs.

16. The system of claim 9, the LETD further comprising a high-speed optical switch with a switching time of less than 1 μs.

17. The system of claim 9, the LETD further comprising an optical chopper configured to occlude the aiming beam for more than 99% of each ms during system operation.

18. A method, comprising:
determining a first intensity value based on first reflected laser light corresponding to laser light of a first wavelength, wherein the laser light of the first wavelength exits a distal end of an optical fiber and the first reflected laser light is reflected by a target and enters the distal end of the optical fiber;
determining a second intensity value based on second reflected laser light corresponding to laser light of a second wavelength, wherein the laser light of the second wavelength exits the distal end of the optical fiber and the second reflected laser light is reflected by the target and enters the distal end of the optical fiber;
determining a third intensity value corresponding fluoresced laser light corresponding to laser light of a fourth wavelength, wherein a laser light of a third wavelength exits the distal end of the optical fiber and the fluoresced laser light is excited from the target by the light of the third wavelength and enters the distal end of the optical fiber;
computing a ratio of the first intensity value and the second intensity value;
determining a distance between the distal end of the optical fiber and the target based on the computed ratio; and
determining whether a calculated luminescence of the target is greater than a threshold luminescence based on the determined distance and the third intensity value.

19. The method of claim 18, comprising:
halting a therapeutic laser light of a fifth wavelength in response to determining that the calculated luminescence of the target is less than the threshold luminescence.

20. The method of claim 18, comprising emitting the laser light of the third wavelength for a duration of fewer than 10 μs each ms.
